(19) United States
(12) Patent Application Publication
(10) Pub. No.: US 2024/0100025 A1
(43) Pub. Date: Mar. 28, 2024

(54) Materials and Methods for Treating Viral and Other Medical Conditions

(71) Applicant: Biotronik AG, Buelach (CH)

(72) Inventor: Ronald E. Betts, La Jolla, CA (US)

(21) Appl. No.: 18/273,708

(22) PCT Filed: Dec. 10, 2021
(86) PCT No.: PCT/EP21/85274

§ 371 Date: Jul. 21, 2023

Related U.S. Application Data
(60) Provisional application No. 63/125,178, filed on Dec. 14, 2020.

(51) Int. Cl. A61K 31/436; A61K 9/00

(52) U.S. Cl. CPC A61K 31/436; A61K 9/0019

(57) ABSTRACT

This application relates generally to the field of drug treatment paradigms based on specifically formulated compounds for use in targeted therapy or disease prevention. Specifically, this technology provides for compositions and methods for treating, stabilizing, preventing or delaying disease conditions related to viral infections and other inflammatory conditions.

### RELATED APPLICATIONS

This application is a U.S. national phase application under 35 U.S.C. § 371 claiming priority from International Application No. PCT/EP2021/085274 filed Dec. 10, 2021, which claims the benefit of priority from U.S. Provisional Patent Application No. 63/125,178 filed Dec. 14, 2020.

### FIELD OF THE INVENTION

This application relates generally to the field of drug treatment paradigms based on specifically formulated compounds for use in targeted therapy or disease prevention. Specifically, this technology provides for compositions and methods for treating, stabilizing, preventing or delaying disease conditions related to viral infections and other inflammatory conditions.

### BACKGROUND OF THE INVENTION

Covid-19 (coronavirus disease 2019) is an illness caused by a new coronavirus, formally named severe acute respiratory syndrome coronavirus 2 (SARS-CoV-2). This disease was initially reported to the World Health Organization (WHO) on Dec. 31, 2019. By Mar. 11, 2020, the WHO declared COVID-19 a global pandemic (Cennimo D. J., et al., Medscape Aug. 10, 2020).

Coronaviruses (CoVs) are a group of viruses that usually cause only mild illnesses such as the common cold. However, in addition to SARS-CoV-2, at least two other human coronaviruses have caused severe symptoms in humans; Middle East respiratory syndrome (MERS-CoV) coronavirus and severe acute respiratory syndrome coronavirus (SARS-CoV). All three are enveloped RNA viruses belonging to the genus betacoronavirus. SARS-CoV-2 has a positive-sense, single stranded RNA genome of approximately 30 kb sharing homology of 80% and 50% with SARS-CoV and MERS-CoV, respectively (Kim D., et al., Cell 181, 2020, 914 921). There are 4 conserved structural proteins common to CoVs: the spike (S) protein, membrane (M) protein, envelope (E) protein and nucleocapsid (N) protein (I. Astuti, Diabetes Metab Syndr 14(4), 2020, 407-412). Initial viral attachment is through the spike S protein targeting host cells (Bosch, et al., J. Virol 77(16), 2003, 8801-8811). SARS-CoV, MERS-CoV and SARS-CoV-2 target respiratory epithelial cells where after binding, viral reproduction and release occurs. This release is followed by viral progression to the lung where acute deleterious response can follow.

Covid-19 statistical tabulations worldwide are difficult due to different testing and reporting methods, data report timing and delay, as well as under-reporting and over-reporting for various political, economic and social reasons. As of Nov. 4, 2020 Covid-19 is affecting 213 countries and territories worldwide with over 48 million cases reported and over 1.2 million deaths (https://www.worldometers.info/coronavirus/). The economic, political and humanitarian impacts of Covid-19 are enormous. For comparative purposes the 2003 SARS-CoV outbreak resulted in approximately 10,000 cases of which 10% died. A retrospective analysis of national statistics of the economic impact of SARS-CoV, a largely Asian outbreak, which affected only the first three quarters of 2003 was approximately $75 billion (Keogh-Brown M. R., et al., Health Policy 88, 2008, 110-120). Detailed SARS Cov-2 epidemiology and conventional viral prevention strategies are beyond the scope of this report.

SARS-CoV-2 therapeutic approaches have centered on two areas; vaccines and drug treatment. Enormous world-wide efforts are now directed toward vaccine development by academic, commercial and other non-profit organizations utilizing a variety of scientific approaches, and while there is shared optimism toward newly started human clinical trials, as of August 2020, there have been no successful vaccines for coronavirus(s) (see https://www.mayoclinic.org/diseases-conditions/coronavirus/in-depth/coronavirus-vaccine/art-20484859 accessed 08182020). At present, there are no drug therapies to either prevent or treat Covid-19 that have been approved by the U.S. Food and Drug Administration (https://www.cdc.gov/coronavirus/2019-ncov/hcp/therapeutic-options.html accessed 08022020). (Note: On Oct. 22, 2020 the U.S. FDA approved remdesivir (Veklury®) injectable for Covid-19 patients requiring hospitalization).

Covid-19 drug approaches can be broken down into two classifications; drugs for prevention and drugs for treatment. Within these two broad classifications both types can contain new active pharmaceutical ingredients (APIs) or repurposed APIs. Because of quickened world response desires, repurposing existing antiviral drugs has received large scale attention. Potential information sources for repurposing drugs include reviewing published literature citing various drug profiles and biological response versus the current CoVs and SARS-CoV-2 genetic, biochemical and pathological knowledge as well as previously conducted pharmaceutical clinical trial results. Worldwide several APIs are being tested/considered for Covid-19 utilization.

It has been observed clinically that, frequently, the most severely or terminally ill SARS CoV and MERS-CoV patients only exhibited mild manifestations during early onset of the disease. The condition of these patients deteriorated suddenly during later stages, or even through recovery periods, with onset of a condition termed acute respiratory distress syndrome (ARDS), resulting in severe lung injury. This injury can also trigger a systemic overflow of inflammatory signaling molecules to other organs and tissues. ARDS is the leading cause of death in SARS-CoV and MERS-CoV patients.

It is accepted that a major causal agent of ARDS in seriously ill CoV patients is the dysregulated and excessive immune response resulting in hyper-production of proinflammatory cytokines, known as a “cytokine storm” or Cytokine Release Syndrome (CRS). Cytokine release syndrome (CRS) is an acute systemic inflammatory response syndrome characterized by fever with or without multiple organ dysfunction. A cytokine is a general term used for a variety of cell signaling molecules. A chemokine is a specific cytokine that functions by attracting cells to sites of infection or inflammation. These signaling molecules play an important role in normal immune responses, but having excess amounts released in the body all at once frequently results in systemic multiple organ failure. Cytokine storm is the term used to denote this severe immune reaction (more information can be found at www.cancer.gov/publications/dictionaries/cancer-terms/def/cytokine-storm accessed).

The underlying cytokine storm present with severe SARS-CoV and MERS-CoV ARDS patients is also common to SARS-CoV-2 viral disease. ARDS is the leading cause of death with these patients (Ye Q., et al., J Infection 80, 2020, 607-613). As with CoVs, a cytokine storm can occur with influenza H1N1 patients and also the result of other pulmonary diseases. Ebola virus disease is also known to trigger dangerous cytokine release (Younan P. et al., mBIO 8(5), 2017, 1-20). Various autoimmune conditions, bacterial sepsis, asthma, allergies, infections, cancer and cancer treatments have additionally been linked with dysregulated cytokine production. It has further been reported that CRS can be triggered by infections or be associated with drugs such as monoclonal antibodies (e.g., rituximab), conventional chemotherapy, immunotherapies with chimeric antigen receptor T (CAR T) cells or Immune Checkpoint Inhibitors such as nivolumab. In addition various other conditions can trigger CRS such as a primary HIV infection, human traumatic brain injury or tissue injury cause by mechanical ventilation. Rapid and effective suppression of excessive cytokine release can greatly reduce patient morbidity and improve outcome. Newly issued international guidelines for the management of sepsis and septic shock, a healthcare problem with more advanced current understanding, indicate that IV administration of antimicrobials be within one hour of recognition and that each hour of delay is associated with measurable increase in mortality (Rhodes A., et al., Crit Care Med 45(3), 2017, 486-552). It is apparent that rapid suppression of viral cytokine release triggering would also be of optimum patient benefit.

Rapamycin, also known as sirolimus, was discovered as a potential antibiotic produced by a Streptomyces species contained in a soil sample collected from Easter Island. In the ensuing years, rapamycin was found to possess potent immunosuppressive and antiproliferative properties in mammalian cells. These unique properties stimulated interest in development of the molecule as a drug substance as well as investigations in mode of action (Li J., Cell Metab 19, 2014, 373-379). This research ultimately resulted in a basic understanding of rapamycin's unique ability to bind and modify a complex assembly of regulatory proteins which have collectively become known as mammalian Target of Rapamycin or mTOR (Sabatini D. M., PNAS (114)45, 2017, 11818-11825).

mTOR is an evolutionarily conserved kinase pathway that has been found to have physiological involvement in a myriad of eukaryotic cell, tissue, organ and system functions and control (Laplante M., et al., J Cell Sci (112)20, 2009, 3589-3894, Saxton R. A., et at., Cell 168, 2017, 1960-1976) Importantly, rapamycin has been shown to be a potent negative effector of mTOR mediated pathways resulting in the development and commercial success as a beneficial regulator of mTOR in a variety of applications. These applications include as an immune suppressor for organ transplant patients, an antiproliferative agent for restenosis control in combination with cardiovascular devices, a topical tuberous sclerosis treatment, certain oncology applications and a key research drug substance for various biochemical investigations. Rapamycin inhibits T-cell proliferation and also inhibits proliferative responses induced by several cytokines, including interleukin 1 (IL-1), IL-2, IL-3, IL-4, IL-6, IGF, PDGF and colony-stimulating factors (CSFs) (Sandrine, F., et al., Nat Rev Drug Discov 5, 2006, 671-688). Rapamycin research activities continue at a rapid pace worldwide. These activities have resulted in the development and commercialization of semi-synthetic derivatives of rapamycin with improved or tailored physical, chemical or biochemical properties specific for the intended use.

It was shown in a small clinical study of hepatitis C positive renal transplant patients that sirolimus was able to suppress hepatitis C (HCV) viral replication. However, the mechanism of this suppression was not identified. (Soliman A., et al., Exp Clin Transplant 5, 2013, 408-411) Additional in vitro investigations were able to demonstrate that, in the case of HCV, sirolimus was able to block viral RNA replication by suppressing the mTORC 1 component of the host cell mTOR protein complex (Stohr S., et al., Gut 65, 2016, 2017-2028). Suggestions were made that this provided rational for further evaluation of mTOR inhibiting therapeutic strategies.

It was also reported, using a mouse in vivo model of H1N1 influenza, that sirolimus and sirolimus in combination with oseltamivir reduced viral titer and the expression of pro inflammatory cytokines and chemokines (Jia X., et al., Plos Pathog 14(11), 2018, 1-25).

In an H1N1 clinical study, 38 patients with confirmed H1N1 pneumonia on ventilator support were randomized to receive adjuvant corticosteroids either with or without sirolimus 2 mg/d for 14 days. Measured clinical values included PaO2, organ failure assessment score and ventilator time. Blood oxygen levels were statistically superior for the sirolimus group on days 3 and 7. Organ failure score was significantly improved in the sirolimus group and mean duration of ventilator time in that group was 7 days versus 15 days. There also was a rapid clearance of virus in the sirolimus group after 7 days of treatment (Wang C., et al., Crit Care Med 42(2), 2014, 313-321, abstract).

Hantavirus pulmonary syndrome (HPS) is a worldwide severe pulmonary disease with fatality rates up to 45%. HPS is caused from infection of Hantavirus, a negative-sense RNA virus having no specific approved vaccines or antiviral therapeutics. Initial viral entry occurs from the virus targeting host lung microvascular endothelial cells. A research group at the U.S. Center for Disease Control and Prevention (CDC) has reported results from investigating the in vitro treatment of Hanta-infected primary human microvascular endothelial cells with the rapamycin analogue temsirolimus (McNulty S., et al., J Virol 87(2), 2013, 912-922). Among other pertinent results, the study reported a 50 percent reduction in virus titers from drug treatment at 0.05 μM, the lowest concentration studied. The study also demonstrated the direct involvement of host cell mTOR by measuring a reduction of p70S6K phosphorylation, the direct mTOR substrate. These authors suggest that “rapamycin and rapamycin derivatives represent potential therapeutic agents for reducing hantavirus pathogenesis by inhibiting viral replication.” (Id.)

An NIH in vitro investigation of MERS-CoV viral replication utilizing MERS-CoV infected cell cultures has been reported (Kindrachuk J., et al., Antimicrob Agents Chemother, 59(2), 2015, 1088-1099). These authors used kinome analysis, phosphorylation patterns and titer measurements to confirm mTOR pathway signaling responses were specifically modulated due to viral infection with overrepresentation of specific signaling intermediates. Treatment of infected cells with rapamycin resulted in viral reduction by 61% at 10 um and 24% at 0.01 um as measured by plaque reduction assay.

It is now established that a wide variety of viruses target the mammalian host mTOR complex to influence and orchestrate responses optimal for their replication (Le Sage V., et al., Viruses, 8(152), 2016, 1-19). It has been suggested that inhibiting viral interactions with mTOR may offer new strategies for viral infection treatment in the clinic. Importantly, mTOR has now been shown to be a key element in previous CoV diseases. And also, rapamycin has previously been demonstrated as both an effector to both block host pathways needed for viral replication and for the modulation of immune response, resulting in reduction of patient cytokine and chemokine response.

Recently human clinical trials have been announced investigating the use of oral sirolimus (Rapamune) for the treatment of Covid-19. Rapamune is available as an oral solution and tablet. Oral sirolimus suffers from poor bioavailability (˜14%) largely due to extensive first pass metabolism. The maximum whole blood concentration (Cmax) of sirolimus after oral administration is approximately 3 hours. A variety of efforts have been made to improve the bioavailability of sirolimus with these efforts being recently reviewed. (Haeri A., et al., Artif Cells Nanomed Biotechnol (46)sup1, 2017, 1-14) None of these efforts however have resulted in a commercial product. There also are no commercially available, approved sirolimus intravenous (IV) formulations. IV formulations typically achieve Cmax rapidly in minutes.

Despite the advances from the studies described supra, there is still an unmet need for a rapid acting therapeutic agent for Covid-19 treatment, as well as for other viral pathogens and conditions, where rapid dampening of virus titers and cytokine response is highly beneficial. Similarly, there is still a need in the art for a novel therapeutic formulation based that acts both towards reduction of viral replication and reduction of potentially fatal cytokine release. Finally, there is a strong desire to improve upon the oral delivery approach of certain medications in order to deliver quicker and/or higher bioavailability of the therapeutic to patients in need thereof.

### SUMMARY OF THE INVENTION

The present invention provides for methods of formulating rapamycin derivatives comprising providing a macrocyclic triene immunosuppressive compound having the structure:

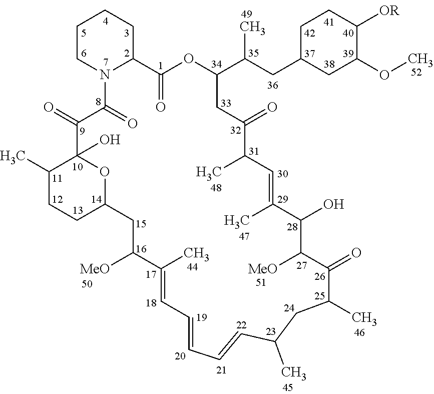

where R is C(O)—(CH₂)ₙ—X, n is 0, 1 or 2, X is a cyclic hydrocarbon having 3-7 carbons, optionally containing one or more unsaturated bonds; providing at least one water soluble solubilizer; mixing the compound with the at least one water soluble solubilizer, wherein the compound is solubilized in the at least one water soluble solubilizer resulting in a drug composition; providing a saline solution; and diluting the drug composition in the saline solution.

Preferably, the water soluble solubilizer is ethyl alcohol (EtOH), propylene glycol, polysorbate, polyethylene glycol 200, 300, 400 or combinations thereof, more preferably the water soluble solubilizer includes up to three substances selected from the group consisting of propylene glycol, polysorbate, polyethylene glycol 200, 300, 400.

In a preferred embodiment, C(O)—(CH₂)ₙ—X has one of the following structures:

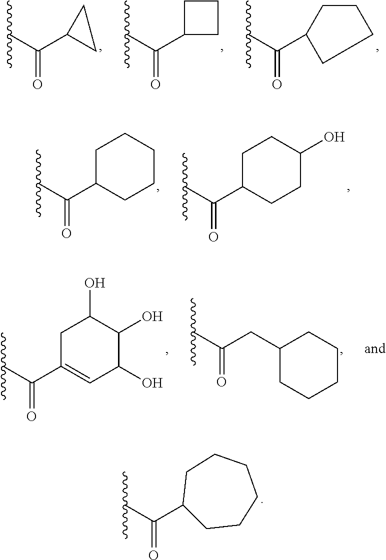

In another aspect, the present invention provides for a method of treating an individual suffering from a condition associated with cytokine release syndrome or a cytokine storm or overproduction of interleukin, the method comprising: administering to the individual a therapeutic amount of a macrocyclic triene immunosuppressive compound having the structure:

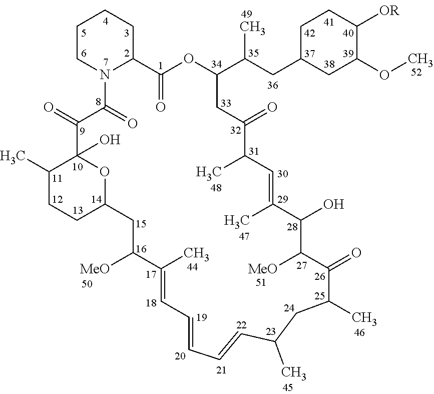

where R is C(O)—(CH₂)ₙ—X, n is 0, 1 or 2, X is a cyclic hydrocarbon having 3-7 carbons, optionally containing one or more unsaturated bonds.

In a preferred embodiment, C(O)—(CH₂)ₙ—X has one of the following structures:

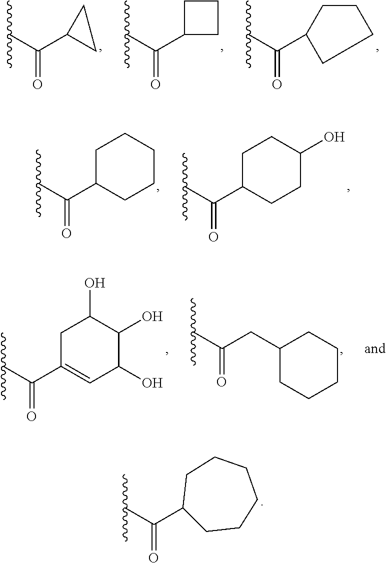

In one aspect the method of treatment is related to a condition of cytokine release syndrome or a cytokine storm or overproduction of interleukin, particularly in the lung. Further, the method of treatment is related to a condition known as Covid-19. Also, the method of treatment is suitable for individuals suffering from sepsis, in particular bacterial sepsis, and further from asthma, allergies, a primary HIV infection, other infections, in particular infections associated with cancer and cancer treatments, conventional chemotherapy, human traumatic brain injury or tissue injury cause by mechanical ventilation, or infections associated with drugs such as monoclonal antibodies (e.g., rituximab), immunotherapies with chimeric antigen receptor T (CAR T) cells or immune checkpoint inhibitors. Further, the method of treatment is suitable for individuals suffering from Covid-19, asthma, infections associated with cancer or cancer treatment in the lung, or tissue injury cause by mechanical ventilation, allergies caused by inhaled allergens.

Also, the present invention provides for a macrocyclic triene immunosuppressive compound having the structure:

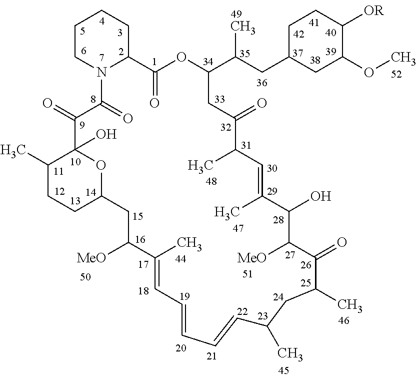

where R is C(O)—(CH₂)ₙ—X, n is 0, 1 or 2, X is a cyclic hydrocarbon having 3-7 carbons, optionally containing one or more unsaturated bonds for use in the treatment of a condition associated with cytokine release syndrome or a cytokine storm or overproduction of interleukin, in particular a cytokine storm or overproduction of interleukin in the lung and further for use in the treatment of Covid-19 or sepsis, in particular bacterial sepsis, and further of asthma, allergies, a primary HIV infection, other infections, in particular infections associated with cancer and cancer treatments, conventional chemotherapy, human traumatic brain injury or tissue injury cause by mechanical ventilation, or infections associated with drugs such as monoclonal antibodies (e.g., rituximab), immunotherapies with chimeric antigen receptor T (CAR T) cells or immune checkpoint inhibitors.

Moreover, the present invention provides for use of a macrocyclic triene immuno-suppressive compound having the structure:

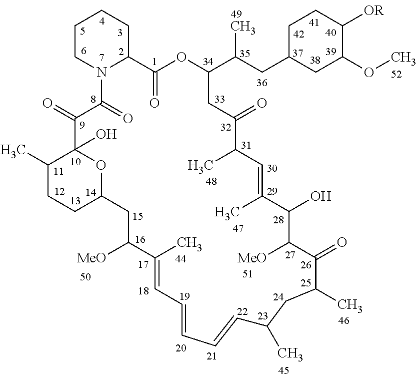

where R is C(O)—(CH₂)ₙ—X, n is 0, 1 or 2, X is a cyclic hydrocarbon having 3-7 carbons, optionally containing one or more unsaturated bonds for the manufacture of a medicament for the treatment of a condition associated with cytokine release syndrome or a cytokine storm or overproduction of interleukin, in particular a cytokine storm or overproduction of interleukin in the lung and further for use in the treatment of Covid-19 or sepsis, in particular bacterial sepsis, and further of asthma, allergies, a primary HIV infection, other infections, in particular infections associated with cancer and cancer treatments, conventional chemotherapy, human traumatic brain injury or tissue injury cause by mechanical ventilation, or infections associated with drugs such as monoclonal antibodies (e.g., rituximab), immunotherapies with chimeric antigen receptor T (CAR T) cells or immune checkpoint inhibitors.

In a further aspect the present invention provides for a method of manufacturing a protein-free drug formulation for parenteral administration comprising (a) providing a macrocyclic triene immunosuppressive compound having the structure:

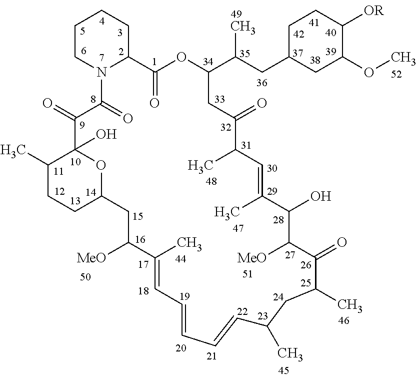

where R is C(O)—(CH₂)ₙ—X, n is 0, 1 or 2, X is a cyclic hydrocarbon having 3-7 carbons, optionally containing one or more unsaturated bonds; (b) providing at least one water soluble solubilizer; (c) mixing the compound with the at least one water soluble solubilizer, wherein the compound is solubilized in the at least one water soluble solubilizer resulting in a drug composition; (d) providing a protein-free saline solution; and diluting the drug composition in the saline solution.

Also, the present invention provides for a protein-free drug formulation comprising a first, a second and a third component, the first component comprising at least one of a macrocyclic triene immunosuppressive compound having the structure:

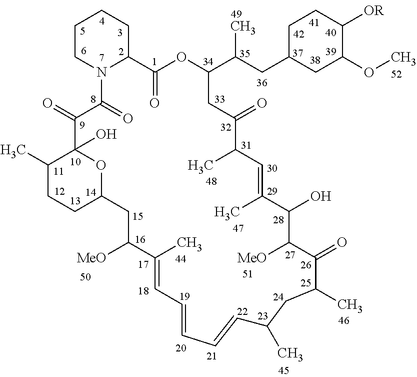

where R is C(O)—(CH₂)ₙ—X, n is 0, 1 or 2, X is a cyclic hydrocarbon having 3-9 carbons, optionally containing one or more unsaturated bonds, the second component comprising at least one protein-free water soluble solubilizer, wherein the first component is solubilized in the second component, and the third component comprising saline.

In a preferred embodiment of the formulation the third component consists of saline.

The present also provides for a kit containing the first, the second and the third components as defined herein in pre-weighed and/or premixed combinations thereof and in sterile container(s) to allow ready parenteral administration.

Preferably, the water soluble solubilizer is ethyl alcohol (EtOH), propylene glycol, polysorbate, polyethylene glycol 200, 300, 400 or combinations thereof.

### BRIEF DESCRIPTION OF THE DRAWINGS

The disclosed subject matter contained herein is best described in conjunction with the accompanying drawings, in which:

FIG. 1 depicts results from a pH stability study comparing CRC-015, sirolimus and everolimus in 0.1M PBS, pH=10 at 37° C.

FIG. 2 shows CRC-015 lipophilicity results at localized tissues as compared to everolimus. Results shown in ElogPoet units.

FIG. 3 shows results of derived ¹⁴C content from male rats administered CRC-015 in each of 25 tissues/organs.

FIG. 4 shows results of derived ¹⁴C content from male rats administered CRC-015 in each of 25 tissues/organs, over time.

FIG. 5 shows area under the curve comparisons (AUC) of CRC-015 and temsirolimus in rat tissues.

FIG. 6 depicts radioactivity concentrations of everolimus in male rat tissues, over time.

### DETAILED DESCRIPTION OF THE INVENTION

As used herein, the term “macrocyclic triene immunosuppressive compound” includes rapamycin (sirolimus), everolimus, zotarolimus, biolimus, novolimus, myolimus, ridaforolimus, temsirolimus and the rapamycin derivatives described in this disclosure.

Conditions associated with cytokine release syndrome or a cytokine storm or overproduction of interleukin include but are not limited to autoimmune diseases, cancer immunotherapy treatments and infections associated with cancer and cancer treatments or conventional chemotherapy, Castleman Disease, sepsis, in particular bacterial sepsis, asthma, allergies, infections associated with human traumatic brain injury or tissue injury caused by mechanical ventilation, or infections associated with drugs such as monoclonal antibodies (e.g., rituximab), immunotherapies with chimeric antigen receptor T (CAR T) cells or immune checkpoint inhibitors and viral diseases such as a primary HIV infection or those related to coronavirus such as Covid-19.

Table 1 summarizes the recent human clinical trials that have been announced investigating the use of oral sirolimus (Rapamune) for the treatment of Covid-19.

TABLE 1
Oral Sirolimus (Rapamune) Human Clinical Trials

| | Number | Sirolimus | |
|---|---|---|---|
| ClinicalTrials.gov | of | (Rapamune) | |
| Identifier | Patients | Dose | Summary of Study |
| NCT04371640 | 40 | Day 1: 10 mg | This is a double-blinded, two-arm, |
| | | Days 2-7: 5 mg | randomized, placebo-controlled study |
| | | | comparing the virological efficacy of add- |
| | | | on sirolimus with standard care to placebo |
| | | | and standard care. Virological efficacy is |
| | | | defined as the change from baseline to day |
| | | | 7 in SARS-COV-2 viral burden measured |
| | | | by quantitative real-time polymerase chain |
| | | | reaction. |
| NCT04341675 | 30 | Day 1: 6 mg | This is a double blind, placebo-controlled |
| | | Days 2-13: 2 mg | study design. 30 subjects will be |
| | | | randomized in a 2:1 fashion to receive |
| | | | sirolimus or placebo. Sirolimus will be |
| | | | given as a 6 mg oral loading dose on day 1 |
| | | | followed by 2 mg daily for a maximum |
| | | | treatment duration of 14 days or until |
| | | | hospital discharge, Chart reviews will be |
| | | | conducted daily to determine changes in |
| | | | clinical status, concomitant medications |
| | | | and laboratory parameters. Study specific |
| | | | biomarkers will be measured at baseline |
| | | | and then at days 3, 7 and 14. |
| NCT04461340 | 40 | Day 1: 6 mg | This is a single blinded randomized clinical |
| | | Days 2-10: 2 mg | trial. 20 patients will get Covid 19 standard |
| | | | of care plus sirolimus. 20 patients will get |
| | | | Covid 19 standard care only. Primary |
| | | | outcome measures are: |
| | | | Time to clinical recovery- |
| | | | [Time Frame: 14-28 days] |
| | | | Viral clearance- |
| | | | [Time Frame: 14 days] |
| NCT04482712 | 20 | 1 mg Daily up to | This is a double blind, placebo-controlled |
| | | 4 Weeks | study design. The primary outcome is to |
| | | | measure survival rate at 4 weeks. |

In the present invention, the stability of the protein-free drug formulation depends on the combination of a target compound together with a water soluble solubilizer. The target compound may be a macrocyclic triene immunosuppressive compound selected from the group consisting of rapamycin (sirolimus), everolimus, zotarolimus, biolimus, novolimus, myolimus, ridaforolimus, temsirolimus and derivatives related thereto. Preferably, the macrocyclic triene immuno-suppressive compound of the present invention is a rapamycin 40-ester analog having the following structure:

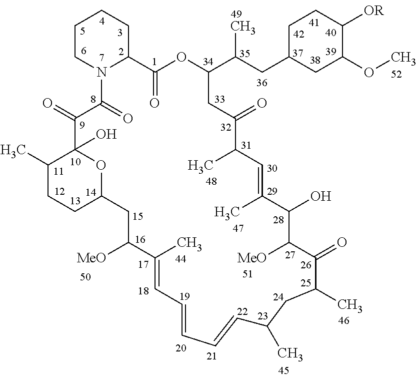

where R is C(O)—(CH₂)ₙ—X, n is 0, 1 or 2, X is a cyclic hydrocarbon having 3-7 carbons and optionally contains one or more unsaturated bonds. In a most preferred embodiment, C(O)—(CH₂)ₙ—X has one of the following structures:

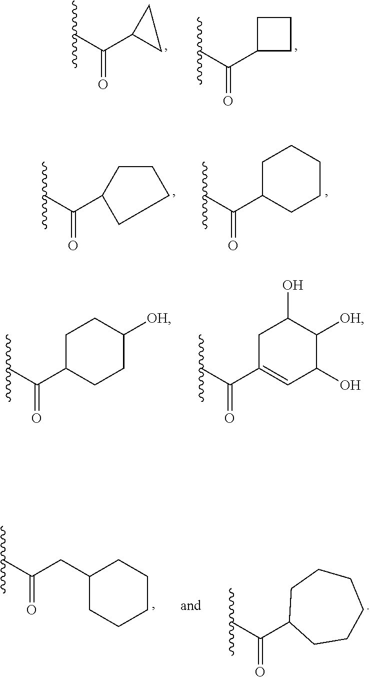

The second component may be a water soluble solubilizer. In a preferred embodiment, the water soluble solubilizer is ethyl alcohol (EtOH), propylene glycol, polysorbate, polyethylene glycol 200, 300, 400 or combinations thereof. In another preferred embodiment the water soluble solubilizer includes up to three substances selected from the group consisting of propylene glycol, polysorbate, polyethylene glycol such as PEG 200, 300 or 400. Preferably, the formulation is first developed by combining the target compound with the water soluble solubilizer, then diluted in a saline solution.

In another embodiment, the at least one water soluble solubilizer of the IV stock solution exists in a ratio of from 100 percent to at least approximately 5 percent of the total solution liquid content and any remaining liquid percentage amount is composed of a second or more water soluble solubilizer combined. The macrocyclic triene immunosuppressive compound content of the IV saline dosing solution is adjusted from approximately 0.05 mg/mL to approximately 60 mg/mL based on dosing requirements.

Additionally, the present invention provides for a method of manufacturing a protein-free drug formulation comprising: (a) providing a macrocyclic triene immunosuppressive compound having the structure:

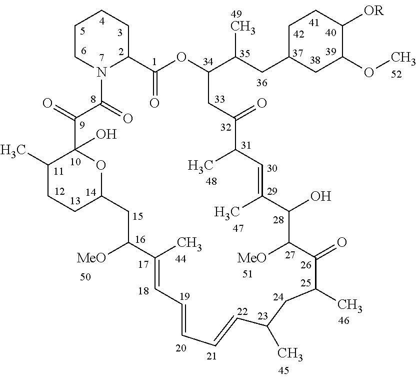

where R is C(O)—(CH₂)ₙ—X, n is 0, 1 or 2, X is a cyclic hydrocarbon having 3-7 carbons, optionally containing one or more unsaturated bonds; (b) providing at least one water soluble solubilizer; (c) mixing the compound with the at least one water soluble solubilizer, wherein the compound is solubilized in the at least one water soluble solubilizer resulting in a drug composition; (d) providing a protein-free saline solution; and diluting the drug composition in the saline solution.

In a preferred embodiment the water soluble solubilizer is ethyl alcohol (EtOH), propylene glycol, polysorbate, polyethylene glycol 200, 300, 400 or combinations thereof.

Also, preferably the water soluble solubilizer includes up to three substances selected from the group consisting of propylene glycol, polysorbate, polyethylene glycol 200, 300, 400.

In one embodiment C(O)—(CH₂)ₙ—X has one of the following structures:

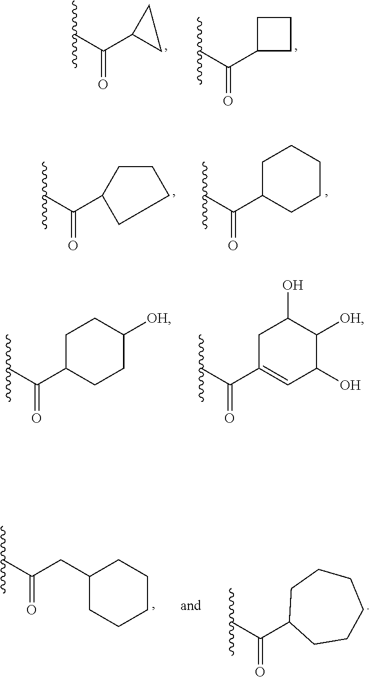

In one aspect of the method described herein to the drug composition a rapamycin derivative selected from the group consisting of sirolimus, everolimus, zotarolimus, biolimus, novolimus, myolimus, ridaforolimus and temsirolimus may be added.

Also, the present invention provides for a protein-free drug formulation comprising a first, a second and a third component, the first component comprising at least one of a macrocyclic triene immunosuppressive compound having the structure:

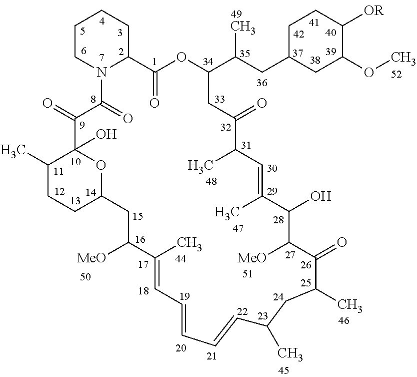

where R is C(O)—(CH₂)ₙ—X, n is 0, 1 or 2, X is a cyclic hydrocarbon having 3-9 carbons, optionally containing one or more unsaturated bonds, the second component comprising at least one protein-free water soluble solubilizer, wherein the first component is solubilized in the second component, and the third component comprising saline.

In a preferred embodiment of the formulation the third component consists of saline.

It has been found with advantage that the formulation of the present invention is protein-free. One of the major problems with distributing a vaccine for Covid-19 will be an uninterrupted cooling of the compounds, because vaccines tend to degradation at ambient or even low temperature up to 10° C. due to protein denaturation. The formulation as defined herein does not suffer from such a disadvantage as the protein-free formulation does not require intense and long-term cooling. Also, the protein-free formulation is easier and cheaper in production and less susceptible to microbial contamination during manufacture and use.

It is therefore also advantageous if the amount of substances introduced to the patient is limited by the administration of the formulation as defined herein. Therefore, it is preferred that the formulation of the present invention consists of a macrocyclic triene immunosuppressive compound having the structure:

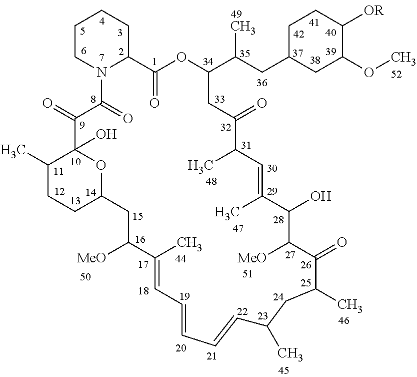

where R is C(O)—(CH₂)ₙ—X, n is 0, 1 or 2, X is a cyclic hydrocarbon having 3-9 carbons, optionally containing one or more unsaturated bonds as a first component, a water soluble solubilizer as a second component having not more than two substances of the group consisting of propylene glycol, polysorbate and polyethylene glycol such as PEG 200, 300 or 400, and as a third component saline.

The present also provides for a kit containing the first, the second and the third components as defined herein in pre-weighed and/or premixed combinations thereof and in sterile container(s) to allow ready parenteral administration.

Also, the present invention provides for a macrocyclic triene immunosuppressive compound having the structure:

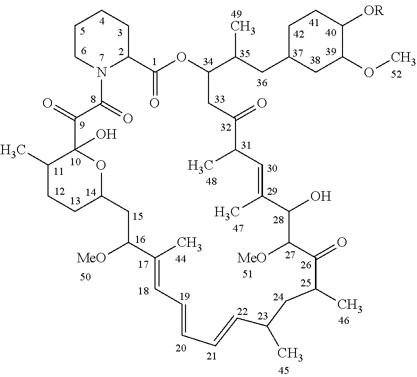

where R is C(O)—(CH₂)ₙ—X, n is 0, 1 or 2, X is a cyclic hydrocarbon having 3-7 carbons, optionally containing one or more unsaturated bonds for use in the treatment of a condition associated with cytokine release syndrome or a cytokine storm or overproduction of interleukin, in particular a cytokine storm or overproduction of interleukin in the lung and further for use in the treatment of Covid-19 or sepsis, in particular bacterial sepsis, and further of asthma, allergies, a primary HIV infection, other infections, in particular infections associated with cancer and cancer treatments, conventional chemotherapy, human traumatic brain injury or tissue injury cause by mechanical ventilation, or infections associated with drugs such as monoclonal antibodies (e.g., rituximab), immunotherapies with chimeric antigen receptor T (CAR T) cells or immune checkpoint inhibitors.

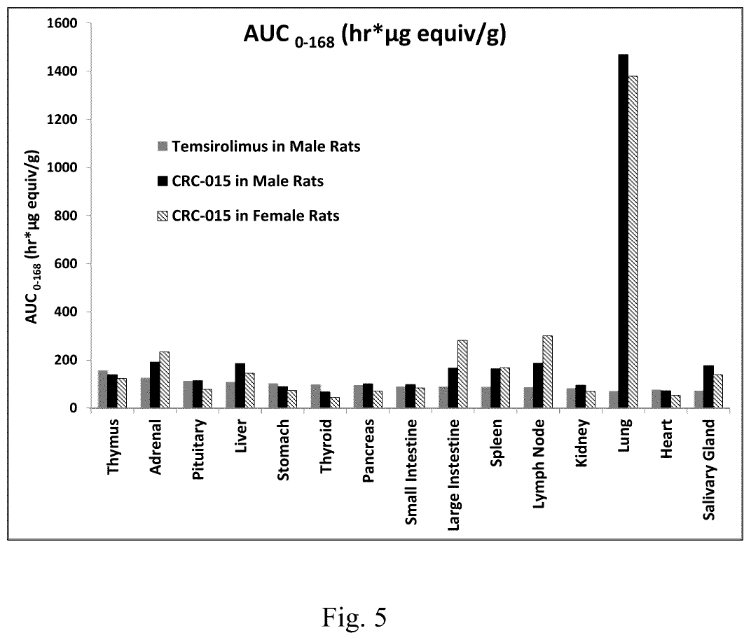

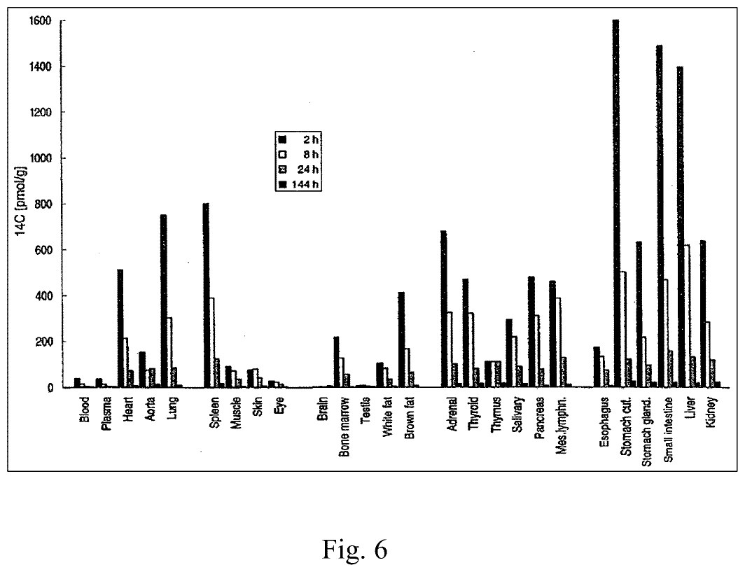

It has been surprisingly found that the macrocyclic triene immunosuppressive compound as defined herein shown unusual accumulation in lung tissue once administered. This is different to other limus compounds which rather accumulated in the glandular tissue or digestive tissue of stomach, intestine, liver or kidney as might be expected (FIGS. 5 and 6). The macrocyclic triene immunosuppressive compound in contrast accumulates in the lung which predestines the herein defined macrocyclic triene immuno-suppressive compound for conditions related to the lung, in particular if the condition requires fast treatment such as in a case of Covid-19, but also conditions requiring long term treatments such as lung cancer. In connection with the long-term stability in basic, physiological conditions the macrocyclic triene immunosuppressive compound as defined herein have high potential as medicament in the treatment of conditions related to cytokine storms such as Covid-19 or sepsis, as the compound will remain available in the blood much longer as related limus derivatives.

### EXAMPLES

### Example Formulations

The macrocyclic triene immunosuppressive compound of the present invention has more than one embodiment and may be described as comprising at least one of the following species from Table 2:

TABLE 2
Description of CRC-015 species

| | R is C(O)— | |
|---|---|---|
| | (CH₂)n—X having | |
| | one of the | |
| | following | |
| Main structure | structures | Species |
| 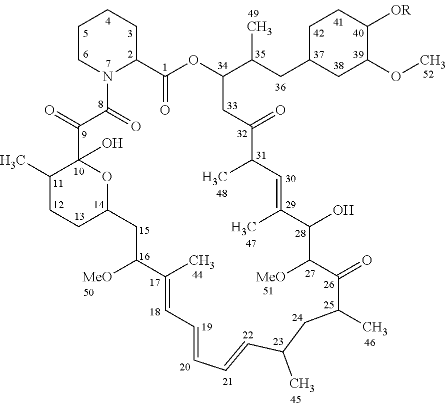 | 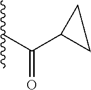 [SMILES: *C(=O)C1CC1] | CRC-015a |
| | 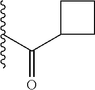 [SMILES: *C(=O)C1CCC1] | CRC-015b |
| | 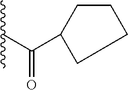 [SMILES: *C(=O)C1CCCC1] | CRC-015c |
| | 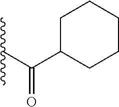 [SMILES: *C(=O)C1CCCCC1] | CRC-015d |
| | 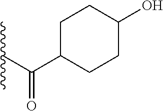 [SMILES: *C(=O)C1CCC(O)CC1] | CRC-015e |
| | 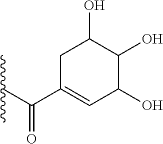 [SMILES: *C(=O)C1=CC(O)C(O)C(O)C1] | CRC-015f |
| | 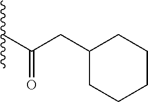 [SMILES: *C(=O)CC1CCCCC1] | CRC-015g |
| | 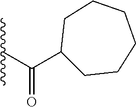 [SMILES: *C(=O)C1CCCCCC1] | CRC-015h |

CRC-015 is a term meant to encompass a genus and used to refer to each of the following species from Table 2: CRC-015a, CRC-015b, CRC-015c, CRC-015d, CRC-015e, CRC-015f, CRC-015g, and CRC-015h.

### I. Mode of Action/Drug Stability Studies of CRC-015:

CRC-015 is a novel, semi-synthetic rapamycin derivative being developed for vascular and other patient uses.

A mode of action study utilizing an ELISA-based assay (K-LISA™) was used to compare mTOR inhibition by CRC-015 to that of rapamycin and everolimus. This assay, unlike simple competitive binding assays, determines the ability to inhibit a functioning mTOR kinase toward the natural substrate, p70S6K. The test kit utilizes human recombinant binding proteins FKBP12 and mTOR as well as human recombinant mTOR substrate P70SK6 and was conducted per the vendor's instructions for use including test controls. An additional control, HPLC examination of CRC-015 assay mixtures, was conducted to ensure that no hydrolysis of CRC-015 to rapamycin had occurred under the conditions of the test.

By comparing the inhibition of phosphorylated p70S6K production it was determined that inhibitory CRC-015 concentrations were similar and not statistically different from sirolimus. Therefore, chemical modification of rapamycin to produce CRC-015 did not reduce, in vitro, mTOR inhibition ability. Intracellular in vivo mTOR inhibitory activity of CRC-015 undoubtedly proceeds through a mechanism similar to that of rapamycin.

Although CRC-015 in vitro mTOR inhibitory effect appears to be similar to rapamycin, it is apparent the specific CRC-015 structural modifications of rapamycin have imparted other distinctive in vitro and in vivo chemical and biochemical properties, some of which are shown below:

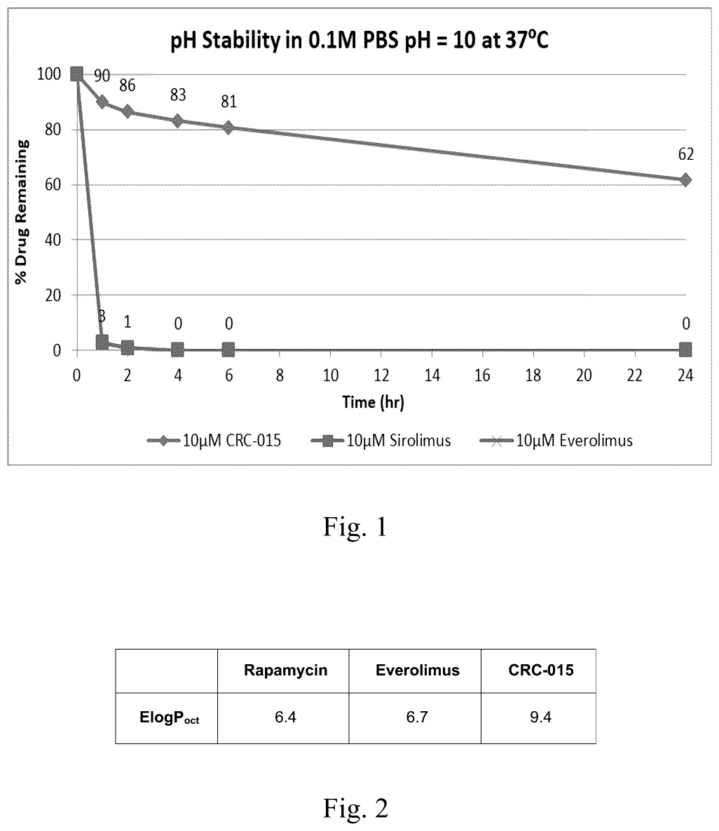

Drug pH stability comparison of CRC-015, sirolimus, and everolimus was conducted in phosphate buffered saline (PBS) under accelerated pH 10 (37° C.) conditions. Sirolimus and everolimus were determined to have similar drug stabilities exhibiting rapid drug degradation rates that occur within 2 hours. CRC-015 was found to have a unique chemical property that results in far greater basic pH stability (FIG. 1).

### II. Protein Binding/Lipophilicity Properties of CRC-015:

CRC-015 has protein binding properties slightly higher than sirolimus with a measured value of 98.1% versus 95.1% (data not shown).

CRC-015 was found to have increased lipophilicity for enhanced performance at localized tissues (FIG. 2).

### III. Parenteral Formulations of CRC-015:

A parenteral formulation of CRC-015 has been developed. The two-part formulation consists of an intravenous (IV) concentrate which is diluted into injectable saline for patient infusion. Both the IV concentrate and IV solution have been shown to have acceptable stability when stored and used per the manufacturer's instructions for use.

The pharmacokinetics, tissue distribution, and mass balance of [¹⁴C]-CRC-015 derived radioactivity following a single intravenous dose in experimentally naive male and female Sprague-Dawley rats was assessed. This study was conducted and reported using good laboratory practice in compliance with the United States Food and Drug Administration (FDA), Department of Health and Human Services, Title 21 Code of Federal Regulations Part 58. Treatment of the animals (including but not limited to all husbandry, housing, and feeding conditions) was conducted in accordance with the guidelines recommended in Guide for the Care and Use of Laboratory Animals (National Academy Press, Washington DC, 2011). Housing conformed to the USDA Animal Welfare Act (9 CFR Parts 1, 2, and 3).

The overall methods and dose level were selected based on available FDA published data to allow comparative analysis of the two active ingredients. CRC-015 test animals therefor received a single bolus, 2.5 mg/kg intravenous dose of the [¹⁴C]-test article. This administration route is also consistent with a proposed route of administration in humans. Macrocycle ring ¹⁴C labeled [¹⁴C]-CRC-015 preparation, chemical analysis and quality control were conducted per accepted industry practice.

A study to determine CRC-015 tissue distribution in rats after IV infusion using this formulation has been completed using ¹⁴C-labelled CRC-015 ([¹⁴C]-CRC-015). This GLP study was conducted in a manner consistent with methods reported by Wyeth. (see Report RPT-44598, CCl-779: tissue distribution of [¹⁴C]-CCI-779-derived radioactivity following a single 2.5 mg/kg intravenous dose of [¹⁴C]-CCI in male Sprague-Dawley and Long-Evans rats, https://www.accessdata.fda.gov/drugsatfda_docs/nda/2007/022088s000_PharmR.pdf at p. 38-40, accessed 08/30/2020) CCI-779 is the Wyeth code identifier for temsirolimus.

Identical to the Wyeth temsirolimus study, [¹⁴C]-CRC-015 dosing was 2.5 mg/kg and administered as a bolus via the tail vein. This was followed by whole blood and specific tissue drug radioactivity measurements at the same time points as in the Wyeth study for pharmacokinetic comparisons of the two compounds. The CRC-015 investigation also substituted female Sprague-Dawley rats for the male pigmented Long-Evans rat temsirolimus study arm. The ¹⁴C label for both CCI-779 and CRC-015 is contained in the rapamycin ring.

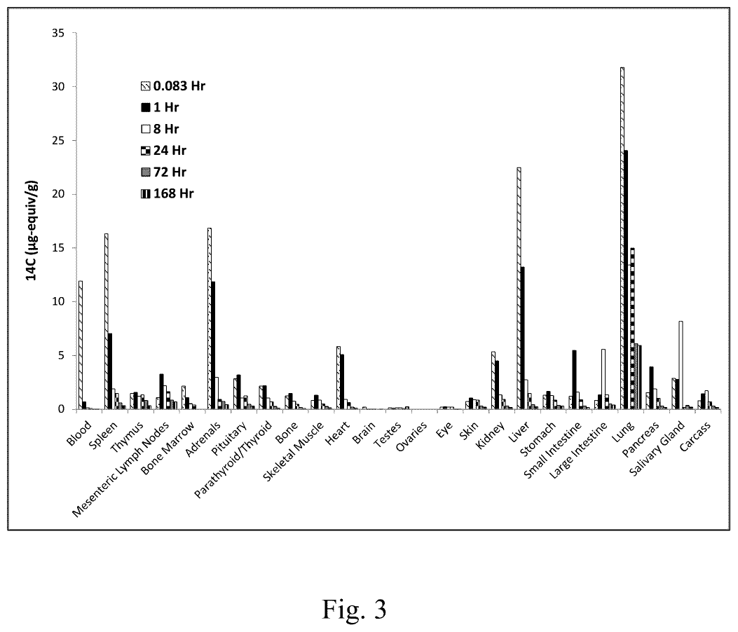

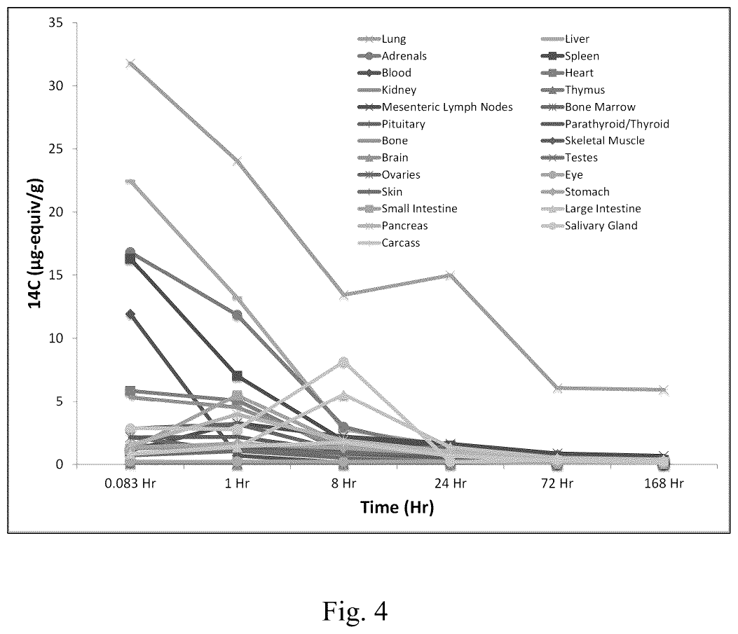

Results of derived ¹⁴C content from male rats administered CRC-015 in each of 25 tissues/organs over time are shown (FIGS. 3 and 4). Female rats had similar results. Results are also shown for area under the curve comparisons (AUC) of CRC-015 and temsirolimus (FIG. 5). More detailed CRC-015 pharmacokinetic measurements are shown below in Table 3.

TABLE 3
Tissue Total Radioactivity Pharmacokinetic Parameters of CRC-015 in Male Rats

| | | Pharmacokinetic Parameter | | | | |
|---|---|---|---|---|---|---|
| | | | | | AUC₀₋₁₆₈ | AUC_{0-∞} |
| | | Cmax (μg- | | Half-life | (hr* μg- | (hr* μg- |
| Tissue Type | | equiv/g) | Tmax (hr) | (hr) | equiv/g) | equiv/g) |
| Vascular/Lymphatic | Blood | 11.916 | 0.083 | NR¹ | 15.880 | NR |
| | Spleen | 16.306 | 0.083 | 62.8 | 163.576 | 192.381 |
| | Thymus | 1.556 | 1 | 72.3 | 138.354 | 173.179 |
| | Mesenteric Lymph Nodes | 3.262 | 1 | 125 | 185.822 | 308.708 |
| | Bone Marrow | 2.145 | 0.083 | 16.7 | 23.086 | 23.147 |
| Endocrine | Adrenals | 16.822 | 0.083 | 126 | 191.217 | 266.946 |
| | Pituitary | 3.191 | 1 | 77.0 | 113.734 | 145.493 |
| | Parathyroid/Thyroid | 2.175 | 1 | 57.4 | 66.946 | 76.062 |
| Skeletal/Muscular | Bone | 1.473 | 1 | 56.8 | 43.983 | 49.806 |
| | Skeletal Muscle | 1.311 | 1 | 85.1 | 56.317 | 73.994 |
| | Heart | 5.849 | 0.083 | 49.2 | 72.374 | 78.907 |
| CNS | Brain | 0.202 | 0.083 | 55.6 | 3.460 | 3.941 |
| Reproductive | Testes | 0.234 | 168 | NC² | 21.485 | NC |
| | Ovaries | NA³ | NA | NA | NA | NA |
| Ocular | Eye | 0.223 | 1 | 53.9 | 15.548 | 17.805 |
| Adipose | Skin | 1.043 | 1 | 72.3 | 72.789 | 94.169 |
| Excretory/Metabolic | Kidney | 5.328 | 0.083 | 52.6 | 94.538 | 106.380 |
| | Liver | 22.480 | 0.083 | 47.9 | 185.033 | 201.347 |
| Gastrointestinal | Stomach | 1.663 | 1 | NR | 88.536 | NR |
| | Small Intestine | 5.486 | 1 | 52.1 | 97.368 | 109.777 |
| | Large Intestine | 5.569 | 8 | NR | 165.556 | NR |
| Respiratory | Lung | 31.791 | 0.083 | NR | 1468.768 | NR |
| Secretory | Pancreas | 3.951 | 1 | 49.3 | 100.328 | 112.633 |
| | Salivary Gland | 8.165 | 8 | 115 | 146.756 | 179.368 |
| Residual | Carcass | 1.731 | 8 | 74.4 | 75.527 | 92.813 |

NR¹ = Not reported, due to poor goodness-of-fit (R² < 0.8) for the elimination phase.
NC² = Not calculated, due to insufficient data points for the elimination phase.
NA³ = Not applicable; tissue not present in this sex.

The results of a similar tissue distribution study (Novartis study) conducted with everolimus are shown in FIG. 6. Table 4 compares the CRC-015 AUC to that of temsirolimus for the 15 organs/tissues having the largest drug concentrations.

TABLE 4
Comparison of Radioactivity Concentration of Temsirolimus and CRC-015 in Rat Tissues

| | ¹⁴C Temsirolimus | | ¹⁴C CRC-015 | | | |
|---|---|---|---|---|---|---|
| | Male | | Male | | Female | |
| | AUC₀₋₁₆₈ | AUC_{0-∞} | AUC₀₋₁₆₈ | AUC_{0-∞} | AUC₀₋₁₆₈ | AUC_{0-∞} |
| Tissue Type | (hr*μg equiv/g) | (hr* μg equiv/g) | (hr* μg equiv/g) | (hr* μg equiv/g) | (hr* μg equiv/g) | (hr* μg equiv/g) |
| Thymus | 156.5 | 180.6 | 138.4 | 173.2 | 122.6 | 138.3 |
| Adrenal | 125.0 | 131.6 | 191.0 | 266.9 | 233.5 | 253.1 |
| Pituitary | 112.1 | 160.0 | 113.7 | 145.5 | 77.3 | 79.5 |
| Liver | 108.3 | 113.8 | 185.0 | 201.3 | 144.4 | 155.1 |
| Stomach | 102.8 | 131.6 | 88.5 | — | 72.6 | 91.3 |
| Thyroid | 98.4 | 111.7 | 66.9 | 76.0 | 45.0 | 51.8 |
| Pancreas | 95.2 | 97.2 | 100.3 | 112.6 | 71.2 | 78.9 |
| Small Intestine | 89.7 | 96.1 | 97.4 | 109.8 | 83.5 | — |
| Large Intestine | 88.6 | 103.9 | 165.6 | — | 281.3 | 299.0 |
| Spleen | 87.2 | 95.6 | 163.6 | 192.4 | 167.4 | 188.7 |
| Lymph Node | 86.1 | 89.0 | 185.8 | 308.7 | 300.9 | 381.0 |
| Kidney | 82.1 | 86.0 | 94.5 | 106.4 | 68.6 | 72.7 |
| Lung | 70.6 | 72.4 | 1468.8 | — | 1379.6 | 2730.7 |
| Heart | 75.8 | 77.1 | 72.4 | 94.2 | 53.7 | 56.5 |
| Salivary Gland | 72.1 | 75.1 | 176.8 | 178.4 | 139.2 | — |

The obvious standout feature of these tissue distribution and comparison studies is the surprising CRC-015 lung targeting in both male and female rats. This was not observed in the Wyeth temsirolimus, nor the Novartis everolimus study, where in both cases the highest drug concentrations were in the gastrointestinal tract and liver.

### IV. Preparation Methods of the IV Dose Formulation:

The methods of preparation of the IV dose formulation were as follows:

A. Stock Solution—Propylene glycol, USP (PPG): Polysorbate 80, USP (50%/50%).

PPG (5 mL) was transferred to a glass vial. A stir bar was added to the vial, the vial was placed on a stir plate and the PPG was stirred at a low speed. Polysorbate 80 (5 mL) was next transferred to the vial and the contents were mixed by stirring. The PPG/Polysorbate mixture was then filtered through a sterile 0.22-μm PVDF filter into a sterile vial. Into a second sterile vial, isotopically diluted [¹⁴C]-CRC-015 test article was added in appropriate amounts. The filter sterilized PPG/Polysorbate was added to the test article and stirred until the test article dissolved. The target concentration of the isotopically diluted [¹⁴C]-CRC-015 in the Stock Solution was approximately 15-20 mg/mL. This Stock Solution was stored at 2 to 8° C. when not in use.

B. IV Dosing Solution—Stock Solution diluted with Saline for Injection

IV Dosing Solution was formulated by adding an appropriate amount of Stock Solution warmed to ambient temperature to a sterile glass jar containing a sterile stir bar. 0.9% Sodium Chloride for Injection, USP, was added to the Stock Solution to dilute to the final CRC-015 concentration of 0.5 mg/mL. The materials were mixed as necessary and aliquoted to sterile container. The IV Dosing Solution was used within four hours of the completion of preparation.

The radiolabel content in the tissue samples was determined by solubilization followed by liquid scintillation counting using standard means. Data from the analyses of the blood and tissue radioactivity concentration data were used to calculate the pharmacokinetic parameters. Noncompartmental analysis was conducted with WinNonlin, version 6.2, operating as a validated software system.

V. Comparison of CRC-015 to Sirolimus Whole Blood Concentrations after Bolus Intravenous Infusion:

Rapid attainment of patient therapeutic drug levels is highly important in treatment of acute hyperinflammatory disorders. Studies were conducted to compare concentrations of CRC-015 and sirolimus in whole blood one hour after intravenous (IV) administration to Sprague-Dawley rats.

Each of three studies utilized six cannulated animals; three receiving IV CRC-015 and three receiving IV sirolimus. Each of the three studies were done on a separate day.

Stock Solutions of CRC-015 and sirolimus at equal molar drug concentration were prepared in propylene glycol/polysorbate 80 as previously described. IV Dosing Solution was prepared by adding 0.9% sodium chloride for injection (USP) to an appropriate amount of Stock Solution to give a final concentration of 0.756 mg/mL for CRC-015 or 0.676 mg/mL for Rapamycin. The solutions were mixed by vortex and used immediately. Drug boluses were prepared so that each cannulated animal received equal molar IV, 2.44 umol/kg drug amounts. After 1 hour, rats were anesthetized and whole blood was collected via cardiac puncture using a 1 cc syringe with a needle. Whole blood (600 μL) was placed into a centrifuge tube containing heparin and the tube mixed by inversion six times. 500 uL whole blood with heparin was then transferred into a new centrifuge tube containing 100 μL of esterase inhibitor (20 μL 12.5% TFA and 80 μL 0.5 M NaF) and mixed by inversion as for above. The sample was quickly frozen in dry ice and stored at −80° C. until analysis. LC/MS sample analysis was conducted by industry recognized methods including the use of deuterium labeled CRC-015 and sirolimus as internal standards and appropriate standard curves and curve verification. Results of the three separate comparative tests are summarized in Table 5.

TABLE 5
Comparison of whole blood drug concentrations at one-hour time point

| | CRC-015 IV Dosing Formulation | | Rapamycin IV Dosing Formulation | | CRC to Rapamycin | |
|---|---|---|---|---|---|---|
| Experiment | Total Drug | Total Drug | Total Drug | Total Drug | Total Drug Ratio | |
| # | ng/mL | nM | ng/mL | nM | ng/mL | nM |
| 1 | 282 | 282 | 156 | 171 | | |
| 1 | 188 | 190 | 153 | 168 | | |
| 1 | 271 | 271 | 155 | 170 | | |
| 2 | 190 | 193 | 145 | 159 | | |
| 2 | 289 | 291 | 78 | 85 | | |
| 2 | 258 | 259 | 236 | 258 | | |
| 3 | 254 | 252 | 137 | 150 | | |
| 3 | 211 | 211 | 131 | 143 | | |
| 3 | 246 | 245 | 115 | 126 | | |
| Average | 243 | 244 | 145 | 159 | 1.7 | 1.5 |

When animals were dosed with equal molar amounts of drug, at one-hour CRC-015 provided significantly higher whole blood concentrations than sirolimus. CRC-015 can therefore provide higher therapeutic drug levels than equal sirolimus amounts or the same therapeutic level with lower dose amounts. This unexpected CRC-015 property thus allows the clinician to better tune IV patient dosage regime.

Taken together the findings of the referenced manuscripts and presented data result in the following conclusions:

There is an unmet need for a rapid acting therapeutic agent for Covid-19 treatment as well as for other viral pathogens and conditions where rapid dampening of virus titers and hyperinflammatory cytokine response is highly beneficial. There is an unmet need for a rapid acting therapeutic agent for treating hyperinflammatory response in other conditions such as: autoimmunity and autoinflammatory disorders, fungal and bacterial infections, sepsis, CAR-T cell therapy and other cancer treatments, pancreatitis, graft-versus-host disease, multiple sclerosis, multiple organ dysfunction syndrome, and the like.

IV administration of the semi-synthetic sirolimus derivative CRC-015, unlike sirolimus derivatives temsirolimus and everolimus, has been found to target lung tissues in an in vivo rat model study. This targeting delivers high levels of drug to the lungs. SARS-CoV-2 manifests Covid-19 by attacking lung cells.

Oral sirolimus has been suggested for repurposing to treat the Covid-19 pandemic based on findings of biological activity toward both reduction of viral replication and reduction of potentially fatal cytokine release. IV formulations of sirolimus having desirable quicker and/or higher bioavailability as compared to oral sirolimus are not available worldwide.

A CRC-015 IV formulation has now been successfully developed and has been approved for human study. This formulation delivers maximum whole blood drug concentrations in minutes as compared to hours with oral sirolimus. This formulation was utilized in the lung targeting in vivo study.

CRC-015 is potentially triple acting for Covid-19 by targeting the lungs, reducing viral replication and reducing cytokine release.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of ordinary skill in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

1.-13. (canceled)

14. A method of manufacturing a protein-free drug formulation for parenteral administration comprising (a) providing a macrocyclic triene immunosuppressive compound having the structure: 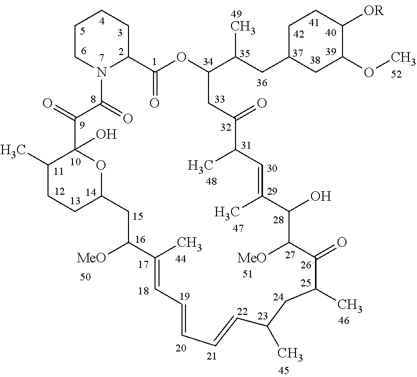
where R is C(O)—(CH₂)ₙ—X, n is 0, 1 or 2, X is a cyclic hydrocarbon having 3-7 carbons, optionally containing one or more unsaturated bonds; (b) providing at least one water soluble solubilizer; (c) mixing the compound with the at least one water soluble solubilizer, wherein the compound is solubilized in the at least one water soluble solubilizer resulting in a drug composition; (d) providing a protein-free saline solution; and diluting the drug composition in the saline solution.

15. The method of claim 14, wherein the water soluble solubilizer is ethyl alcohol (EtOH), propylene glycol, polysorbate, polyethylene glycol 200, 300, 400 or combinations thereof.

16. The method of claim 14, wherein the water soluble solubilizer includes up to three substances selected from the group consisting of propylene glycol, polysorbate, polyethylene glycol 200, 300, 400.

17. The method of claim 14, wherein C(O)—(CH₂)ₙ—X has one of the following structures:

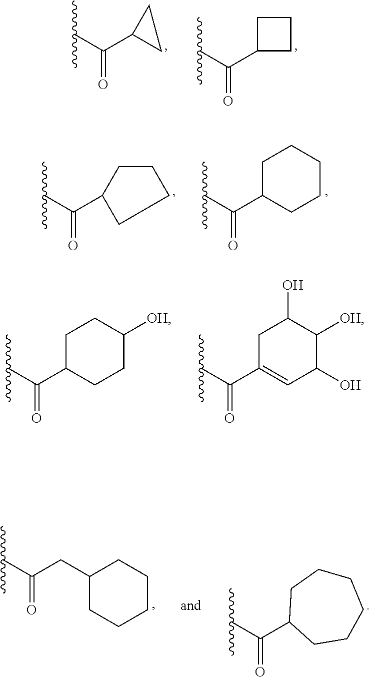

18. The method of claim 14, wherein to the drug composition a rapamycin derivative selected from the group consisting of sirolimus, everolimus, zotarolimus, biolimus, novolimus, myolimus, ridaforolimus and temsirolimus may be added.

19. A method of treating an individual suffering from a condition associated with cytokine release syndrome or a cytokine storm or overproduction of interleukin, the method comprising administering to the individual a therapeutic amount of a macrocyclic triene immunosuppressive compound having the structure: 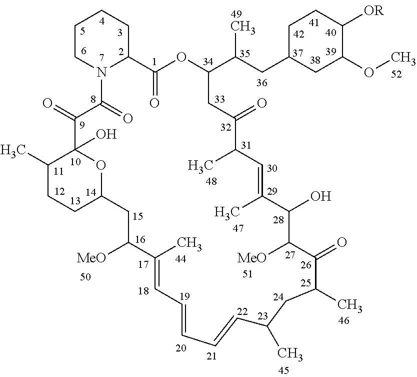
where R is C(O)—(CH₂)ₙ—X, n is 0, 1 or 2, X is a cyclic hydrocarbon having 3-7 carbons, optionally containing one or more unsaturated bonds.

20. The method of claim 19, wherein the condition is autoimmune diseases, cancer immunotherapy treatments and infections associated with cancer and cancer treatments or conventional chemotherapy, Castleman Disease, sepsis, in particular bacterial sepsis, asthma, allergies, infections associated with human traumatic brain injury or tissue injury caused by mechanical ventilation, or infections associated with drugs such as monoclonal antibodies (e.g., rituximab), immunotherapies with chimeric antigen receptor T (CAR T) cells or immune checkpoint inhibitors and viral diseases such as a primary HIV infection or those related to coronavirus such as Covid-19.

21. The method of claim 19, wherein the administering comprises a step of intravenous administration of a formulation prepared by one of claims 1 to 3.

22. A protein-free drug formulation comprising a first, a second and a third component, the first component comprising at least one of a macrocyclic triene immunosuppressive compound having the structure: 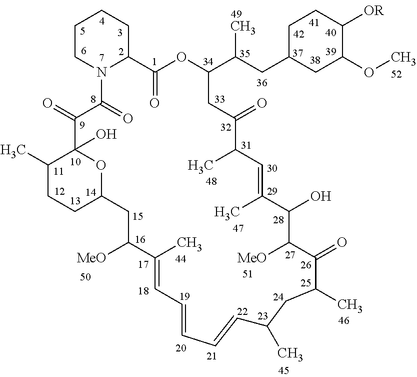
where R is C(O)—(CH₂)ₙ—X, n is 0, 1 or 2, X is a cyclic hydrocarbon having 3-9 carbons, optionally containing one or more unsaturated bonds, the second component comprising at least one protein-free water soluble solubilizer, wherein the first component is solubilized in the second component, and the third component comprising saline.

23. The protein-free drug formulation of claim 22, wherein the third component consists of saline.

24. The protein-free drug formulation of claim 22, wherein the water soluble solubilizer is ethyl alcohol (EtOH), propylene glycol, polysorbate, polyethylene glycol 200, 300, 400 or combinations thereof.

25. The protein-free drug formulation of claim 22, wherein the water soluble solubilizer includes up to three substances selected from the group consisting of propylene glycol, polysorbate, polyethylene glycol 200, 300, 400.

26. The protein-free drug formulation of claim 22, wherein the formulation is a component of a kit, the kit containing the formulation in pre-weighed and premixed combinations thereof and further wherein the formulation is in at least one sterile container to allow ready parenteral administration.
